(19) United States
(12) Patent Application Publication
(10) Pub. No.: US 2008/0009924 A1
(43) Pub. Date: Jan. 10, 2008

(54) LOCALLY HEATING OBJECTS

(76) Inventors: Hans-Herbert Fuchs, Hofheim (DE); Ahmet Cem Firatli, Wiesbaden (DE); Rudolf Majthan, Eschborn (DE)

Correspondence Address: FISH & RICHARDSON PC, P.O. BOX 1022, MINNEAPOLIS, MN, 55440-1022, US

(21) Appl. No.: 11/763,878

(22) Filed: Jun. 15, 2007

(30) Foreign Application Priority Data
Jun. 16, 2006 (DE) 10 2006 027 788.0

(51) Int. Cl. A61F 2/00

(52) U.S. Cl. 607/101

(57) ABSTRACT

The invention pertains to a method for locally heating an object with electromagnetic radiation and substantially simultaneously cooling the immediate vicinity of the object region to be heated by a flowing fluid. The inventive method is characterized in that the flowing fluid and the electromagnetic radiation emitted in the toward the region to be heated are guided along paths that are spatially separated from one another.

### TECHNICAL FIELD

The invention pertains to a method and device for locally heating objects by means of electromagnetic radiation and simultaneously cooling the immediate vicinity of the object region to be heated by means of a flowing fluid.

### BACKGROUND

WO 2004/082428 A2 describes a locally heated toothbrush head. Devices for heating toothbrush heads are described in JP 10118201 A1, JP 10118202 A1 and U.S. Pat No. 4,680,448.

Local heating is used for additional processing of objects that cannot always be manufactured with the desired final contour, particularly plastic parts manufactured by means of injection molding. This is the reason why these objects are initially manufactured with an intermediate contour and then reshaped into the desired final contour in an additional processing step.

In order to purposefully reshape the objects, they are locally heated by means of electromagnetic radiation, and the immediate vicinity of the object region to be heated is simultaneously cooled by means of a flowing fluid. During the local heating of the object, however, other parts of the object that lie in the immediate vicinity of the region to be locally heated may be very heat sensitive and need to remain undamaged. This is achieved by limiting the electromagnetic radiation to certain areas of the object to be locally heated by means of reflectors and diaphragms.

In order to quickly transmit the heat emitted by the electromagnetic radiation, these reflectors and diaphragms lie as close as possible to the object surface to be heated. This also causes the reflectors and diaphragms to heat up over time and to ultimately emit heat. The heating by means of electromagnetic radiation therefore is no longer localized. This is the reason why ambient air is constantly taken in so as to cool the diaphragms and reflectors. The cool air taken in flows past the object region to be heated and the electromagnetic radiation source, as well as through the beam path of the electromagnetic rays on the diaphragms and reflectors, and thus cools these elements. The shielding effect of the diaphragms and reflectors is preserved in this fashion.

However, it was determined that this causes undesirable and irregular cool and hot areas in the region of the reflectors and diaphragms, wherein these cool and hot areas make it difficult to uniformly heat the object to be locally heated and therefore to manufacture such objects, particularly in large quantities.

### SUMMARY

According to a first embodiment of the invention, the flowing fluid and the primary electromagnetic radiation are spatially separated or completely spatially separated (decoupled) from one another. Due to this measure, the cooling effect achieved with the flowing fluid is essentially independent of the radiation source that is cooled separately, if so required.

It was furthermore determined that an efficient cooling process can be achieved by utilizing a fluid in the form of a gas. The utilization of air proved to be a cost-efficient alternative.

According to another embodiment of the invention, the electromagnetic radiation is diffusely guided to the region to be heated. Substantially uniform heating of the local object region is achieved with this measure such that a homogenous structure is present in this region after the manufacture of the object is completed. Inhomogeneities within the structure of the region to be heated could lead to undesirable material properties and are effectively inhibited in this fashion.

According to one aspect of the invention, ultraviolet radiation is used as the electromagnetic radiation for heating the local region. Naturally, it would also be possible to utilize electromagnetic radiation in the form of infrared radiation or laser radiation. The radiation types can be easily and cost-efficiently integrated into an inventive method.

The object to be heated may be irradiated with electromagnetic radiation from at least two sides. This measure additionally increases the homogeneity of the finished product to be manufactured.

The inventive method is suitable for heating plastic objects that, if applicable, are mechanically reshaped during the heating process. The method can be used, in particular, for the manufacture of toothbrush heads.

The fluid flow is essentially blocked from reaching the radiation channel guide opening (aperture) due to the fact that the housing of the device according to the invention features at least one opening leading into the channel in at least one of its housing walls that is arranged essentially perpendicular to the housing opening or radiation channel guide opening. This promotes homogenous electromagnetic radiation for heating the object to be manufactured. This also results in the region to be heated having a substantially homogenous final structure after the object has been cooled. Material defects due to inhomogeneities are inhibited in this fashion.

According to a first embodiment of the inventive device, the opening of the radiation guide channel is coupled to a pane that transmits electromagnetic radiation. This promotes homogenous electromagnetic radiation that uniformly heats the local object region. The formation of inhomogeneities therefore is effectively inhibited.

The pane is realized in the form of a diffuser for the electromagnetic radiation such that the homogeneity of the electromagnetic radiation is additionally increased.

The device may be equipped with different electromagnetic radiation sources. For example, the electromagnetic radiation source may be realized in the form of a laser, a ultraviolet radiation source or an infrared radiation source.

A positioning device is provided in order to position the object in the beam path of the electromagnetic radiation. The objects are fed to the positioning device by means of a feed system. The object to be heated is held in the path of the electromagnetic radiation by the positioning device for an chosen duration, namely with the aid of a timer. After heating and, if applicable, cooling of the object, a delivery device transports the object away from the device.

A microprocessor is provided in order to control and/or program the positioning device and/or the feed system and/or the timer and/or the delivery device.

The invention furthermore pertains to a toothbrush head with at least two sections that can be moved independently of one another by compliant deflection about a connecting region on one end of the toothbrush head, wherein said sections essentially diverge in the shape of a V in an initial contour of the toothbrush head and are transformed into a final contour of the toothbrush head by heating the connecting region as well as mechanically exerting a force upon the open ends of the sections and subjecting the toothbrush head to a subsequent cooling process, and wherein slits of essentially constant width are formed between the sections in the final contour of the toothbrush head. According to the inventive method, the toothbrush head is heated and substantially simultaneously provided with its final contour.

With the flowing fluid spatially separated from the electromagnetic radiation emitted in the direction of the region to be heated, the fluid may no longer have a cooling effect on the region to be locally heated. However, the fluid can still cool the housing of the radiation source as well as the electromagnetic radiation source. This means that the fluid can still fulfill the desired cooling function, particularly on diaphragms and reflectors that may be present. The electromagnetic radiation for heating the object therefore may be stable over time and locally defined along the beam path, as well as limited to an irradiation region and substantially uniform, namely such that it can also be successfully utilized in quantity production.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts a prior art device.

FIG. 2 depicts an embodiment of the invention.

FIG. 3 depicts a toothbrush head before it is treated with an embodiment of the invention in a method according to the invention.

FIG. 4 depicts the toothbrush head of FIG. 3 while it is treated with an embodiment of the invention in a method according to the invention.

FIG. 5 depicts a toothbrush with the toothbrush head of FIG. 4.

Like reference symbols in the various drawings indicate like elements.

### DETAILED DESCRIPTION

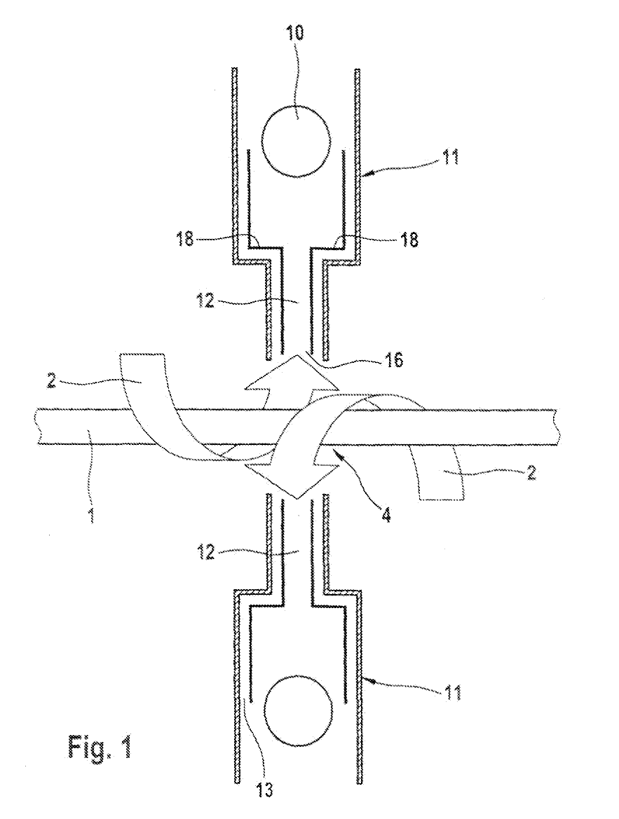

FIG. 1 shows a prior art device. In this case, an object 1 is locally heated in a local region 4 by two radiation sources 10. The radiation sources 10 are arranged in a housing 11. The housing 11 also features guide surfaces 18 that form a radiation guide channel 12, through which the electromagnetic radiation is guided to the local region 4 of the object 1. The guide surfaces 18 feature an opening for the electromagnetic radiation that is guided to the local region 4 of the object 1 through an aperture 16 of the housing 11. The electromagnetic radiation required for heating the local region 4 also heats the guide surfaces 18, the housing 11 and other parts of the device. This causes these parts to emit heat that can lead to undesirable heating of the object 1. Consequently, undesirable structural changes may occur within the object 1 such its material properties may also change, if applicable.

As shown in FIG. 1, a fluid flow 2 that usually consists of an air flow is drawn into the interior of the housing through the housing aperture 16. This flowing fluid cools the heated parts of the device, for example, the guide surface 18, the housing 11, the radiation source 10 and similar parts.

However, this cooling process results in undesirable and irregular cool and hot areas on the radiation source 10 as well as on the radiation shields such as, for example, the guide surfaces 18, wherein undesirable heating of the local region 4 of the object 1 may occur as a result thereof. Such irregular heating is undesirable, particularly for series or quantity production.

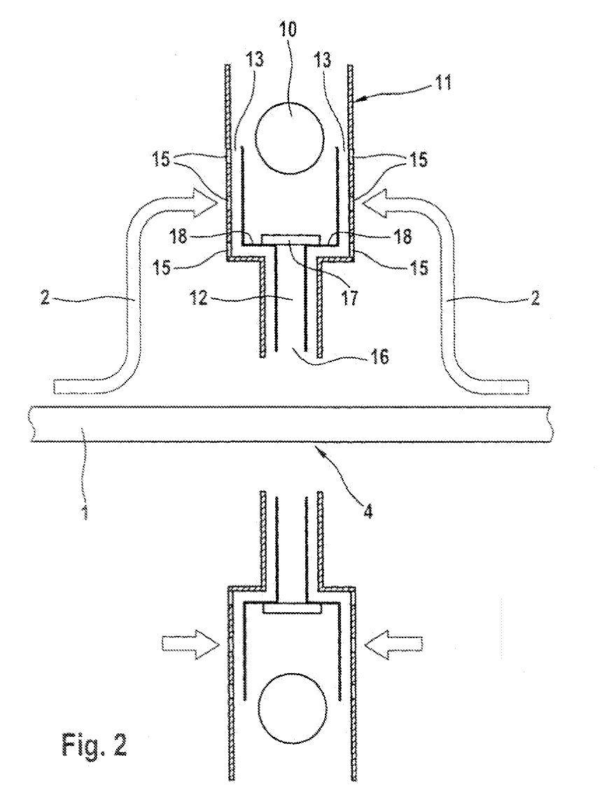

FIG. 2 depicts an embodiment of the invention in which the flowing fluid 2 and the electromagnetic radiation emitted in the direction of the region 4 to be heated are guided along paths that are spatially separated from one another. The paths may be completely spatially separated from one another or decoupled. Openings 15 are provided in the walls of the housing 11 in order to take in the flowing fluid. The flowing fluid is guided along a fluid flow channel 13 such that it is not admitted into the radiation guide channel 12 for the electromagnetic radiation, thereby inhibiting formation of irregular cool and hot areas of the air guide surface 18 within the radiation guide channel 12 and promoting constant and uniform irradiation of the local region 4 of the object 1. Fluid flow channel 13 allows fluid to be conveyed by means of a pump, a fan or the like.

In order to achieve an even more uniform irradiation of the local region 4, a pane 17 is positioned in the radiation guide channel 12 to inhibit the admission of the flowing fluid into the radiation guide channel. The pane 17 allow the radiation to be diffusely guided to the local region 4. This also promotes a homogenous radiation that, in turn, promotes a homogenous structure of the finished object 1.

Objects 1 are typically irradiated from two sides, as illustrated in FIG. 2. However, the reference symbols are only provided on one side of the device in order to provide a better overview.

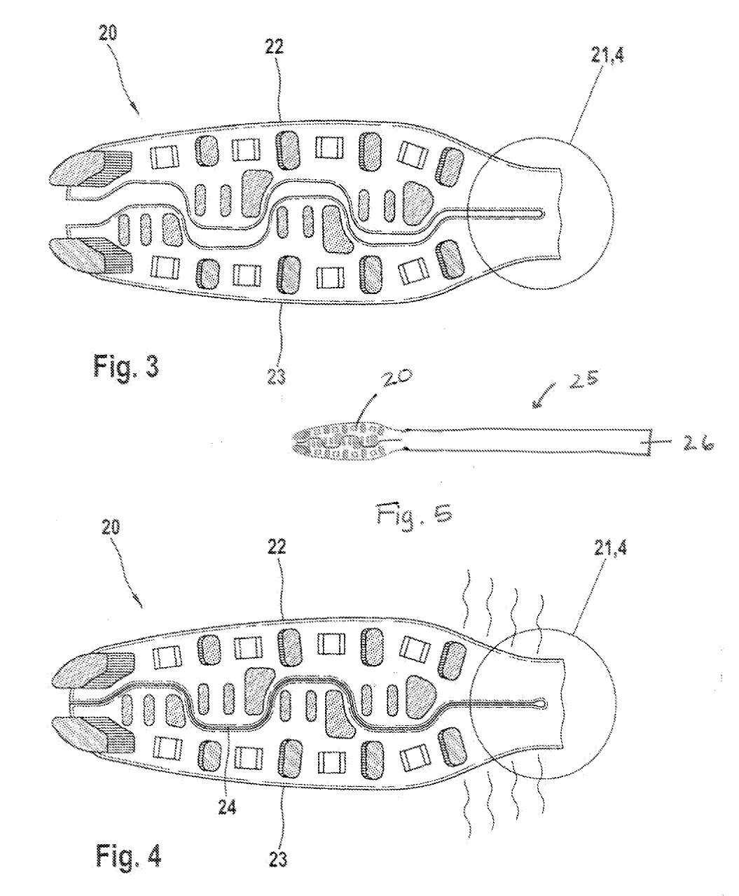

FIGS. 3 and 4 show a toothbrush head that is manufactured in accordance with the inventive method. This toothbrush head 20 is provided with at least two sections 22, 23 that can be moved independently of one another by compliant deflection about connecting region 21 on one end of the toothbrush head 20. The sections 22, 23 essentially diverge in the shape of a V in an initial contour of the toothbrush head 20 that is illustrated in FIG. 3. The toothbrush head 20 is provided with the final contour according to FIG. 4 by heating the connecting region 21, mechanically exerting a force upon the open ends of the sections 22, 23 and subjecting the toothbrush head to a subsequent cooling process. In this final contour, a slit 24 of substantially constant width is formed between the sections 22, 23.

A device according to FIG. 2 is used for heating the connecting region 21 that serves as the local region 4 according to FIG. 2 in this toothbrush.

FIG. 5 depicts a toothbrush 25 with toothbrush head 20 and handle 26.

What is claimed is:

1. A local heating method for locally heating an object, comprising:
heating a region of an object by providing electromagnetic radiation to the region of the object; and
flowing a fluid proximate the heated region to substantially simultaneously cool the object proximate the heated region, and
wherein the fluid and the electromagnetic radiation are guided along paths that are spatially separated from one another.

2. The local heating method of claim 1, wherein the fluid and the electromagnetic radiation are guided along paths that are completely spatially separated from one another.

3. The local heating method of claim 1, wherein the fluid comprises a gas.

4. The local heating method of claim 1, wherein the fluid comprises air.

5. The local heating method of claim 1, wherein the electromagnetic radiation is diffusely guided to the heated region.

6. The local heating method of claim 1, wherein the electromagnetic radiation comprises ultraviolet radiation.

7. The local heating method of claim 1, wherein the electromagnetic radiation comprises infrared radiation.

8. The local heating method of claim 1, wherein the electromagnetic radiation comprises laser radiation.

9. The local heating method of claim 1, wherein providing the electromagnetic radiation to the region of the object comprises irradiating the region of the object from at least two sides.

10. The local heating method of claim 1, wherein the region of the object is a head of a toothbrush.

11. A local heating device for locally heating an object, the device comprising:
a housing defining a first opening and a second opening;
an electromagnetic radiation source coupled to the housing, wherein the electromagnetic radiation source is configured to provide electromagnetic radiation to the object to be heated;
a radiation guide channel coupled to the housing, wherein the radiation guide channel is configured to guide the electromagnetic radiation from the electromagnetic radiation source through the first opening in the housing toward object to be heated; and
a fluid flow channel between the radiation guide channel and the housing, wherein the fluid flow channel is coupled to the first opening and the second opening in the housing, such that fluid allowed to flow into the housing through the second opening exits the housing through the first opening, and wherein the direction of fluid flow into the second opening and out of the first opening are substantially perpendicular.

12. The local heating device of claim 11, wherein the radiation guide channel and the fluid flow channel are spatially separated from one another.

13. The local heating device of claim 11, wherein the radiation guide channel and the fluid flow channel are completely spatially separated from one another.

14. The local heating device of claim 11, further comprising a pane that transmits electromagnetic radiation, wherein the pane is coupled to the radiation guide channel such that electromagnetic radiation guided through the channel and through the first opening in the housing passes through the pane.

15. The local heating device of claim 14, wherein the pane is configured to diffuse the electromagnetic radiation.

16. The local heating device of claim 11, wherein the electromagnetic radiation source comprises a laser.

17. The local heating device of claim 11, wherein the electromagnetic radiation source comprises an ultraviolet radiation source.

18. The local heating device of claim 11, wherein the electromagnetic radiation source comprises an infrared radiation source.

19. The local heating device of claim 11, further comprising a positioning device, wherein the positioning device is operable to position the object in the path of the electromagnetic radiation.

20. The local heating device of claim 19, further comprising a feed system configured to successively feed a plurality of objects to the positioning device.

21. The local heating device of claim 20, further comprising a microprocessor configured to control the positioning device and/or the feed system.

22. The local heating device of claim 19, further comprising a timer, wherein the timer is operable to enable the positioning device to hold the object to be heated in the path of the electromagnetic radiation for a chosen duration.

23. The local heating device of claim 22, further comprising a microprocessor configured to control the timer.

24. The local heating device of claim 11, further comprising a delivery device configured to transport the object away from the electromagnetic radiation source after the object has been heated.

25. The local heating device of claim 24, further comprising a microprocessor configured to control the delivery device.

26. A toothbrush head, comprising:
at least two sections extending from a connecting region of the toothbrush head,
wherein the sections are separated from each other by a slit of substantially constant width,
wherein the sections can be moved independently of one another by compliant deflection about the connecting region of the toothbrush, and
wherein the connecting region of the toothbrush head has been locally heated, wherein local heating comprises:
heating the connecting region of the toothbrush head by providing electromagnetic radiation to the connecting region of the toothbrush head; and
flowing a fluid proximate the heated connecting region to substantially simultaneously cool the toothbrush head proximate the heated connecting region, and
wherein the fluid and the electromagnetic radiation are guided along paths that are spatially separated from one another,
wherein a mechanical force has been exerted upon the sections such that an initial contour of the toothbrush head, in which the sections essentially diverge from the connecting region in the shape of a V, has been shaped into a final contour of the toothbrush head to form the slit of essentially constant width between the sections, and
wherein the toothbrush head has been substantially simultaneously heated and provided with the final contour.

27. A toothbrush comprising a toothbrush head, wherein the toothbrush head comprises:
at least two sections extending from a connecting region of the toothbrush head,
wherein the sections are separated from each other by a slit of substantially constant width,
wherein the sections can be moved independently of one another by compliant deflection about the connecting region of the toothbrush head, and
wherein the connecting region of the toothbrush head has been locally heated, wherein local heating comprises:
heating the connecting region of the toothbrush head by providing electromagnetic radiation to the connecting region of the toothbrush head; and
flowing a fluid proximate the heated connecting region to substantially simultaneously cool the toothbrush head proximate the heated connecting region, and
wherein the fluid and the electromagnetic radiation are guided along paths that are spatially separated from one another,
wherein a mechanical force has been exerted upon the sections such that an initial contour of the toothbrush head, in which the sections essentially diverge from the connecting region in the shape of a V, has been shaped into a final contour of the toothbrush head to form the slit of essentially constant width between the sections, and
wherein the toothbrush head has been substantially simultaneously heated and provided with the final contour.
